(19) United States
(12) Patent Application Publication
(10) Pub. No.: US 2010/0036401 A1
(43) Pub. Date: Feb. 11, 2010

(54) VASCULAR GRAFT AND METHOD OF USE

(75) Inventor: JOSE LUIS NAVIA, SHAKER HEIGHTS, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 12/498,427

(22) Filed: Jul. 7, 2009

Related U.S. Application Data
(60) Provisional application No. 61/079,356, filed on Jul. 9, 2008.

(51) Int. Cl. A61B 17/08

(52) U.S. Cl. 606/155

(57) ABSTRACT

A vascular graft includes an elongated main body portion having a distal end portion defining a first opening and a second proximal end portion defining a second opening. The vascular graft also includes a first sewing ring having a base portion securely attached to the first distal end portion of the elongated body portion, and a second sewing ring having a base portion securely attached to the first distal end portion at a location proximal to the first sewing ring. The first and second base portions form a channel for receiving an end portion of a blood vessel.

### RELATED APPLICATION

This application claims priority from U.S. Provisional Application No. 61/079,356, filed Jul. 9, 2008, the subject matter of which is incorporated herein by reference.

### TECHNICAL FIELD

The present invention relates generally to surgical implants, and more particularly to a vascular graft adapted for anastomosis with at least one blood vessel.

### BACKGROUND OF THE INVENTION

Various surgical prostheses and techniques have been devised to improve the ability to achieve successful anastomosis and reduce the time consumed by anastomosis procedures. For example, tubular prostheses have been inserted into the interior and over the exterior of the vessel which is anastomosed. Such prostheses aid in holding the vessel ends during the procedure and while the vessel tissue grows back together during healing. Some prostheses are made from biological material that is slowly absorbed by the body tissue as healing progresses. Other types of prostheses are made from permanent materials, such as plastics or metals that remain permanently within the interior of the vessel after healing is completed. Still other prostheses incorporate both permanent and biologically absorbable materials that dissolve and are replaced by natural tissue growth.

Biologically dissolvable or absorbable prostheses are sometimes regarded as preferable because no foreign object remains after healing is completed. Such prostheses often present several disadvantages, however, including: partial vessel occlusion and permanent reduction in fluid flow; living tissue rejection of the prostheses; unnatural tissue growth caused by adverse tissue reaction may fully or partially occlude the vessel; and the need for an additional surgical procedure to remove prostheses after anastomosis is complete.

A relatively new procedure for anastomosis involves completely bonding the ends of the vessel together using, for example, a laser beam. Thermal bonding heats the ends of the vessel and creates an inter-linked and cross-linked matrix of dessicated tissue fibers that holds the ends of the vessel together until natural tissue growth occurs. One advantage of thermal bonding is that a continuous bonded “seam” is created to obtain a more complete and leak-free junction of the vessel ends. One disadvantage of thermal bonding, however, is that it requires about the same amount of time to complete as more traditional anastomosis techniques, i.e., surgical suturing. Additionally, the vessel ends must be aligned, abutted, and held together without the aid of metallic clamps (or the like) prior to thermal bonding. Such metallic clamps can divert or deflect the energy beam and cause undesirable localized heating and tissue destruction.

### SUMMARY OF THE INVENTION

According to one aspect of the present invention, a vascular graft comprises an elongated main body portion having a distal end portion defining a first opening and a second proximal end portion defining a second opening. The vascular graft also includes a first sewing ring having a base portion securely attached to the first distal end portion of the elongated body portion, and a second sewing ring having a base portion securely attached to the first distal end portion at a location proximal to the first sewing ring. The first and second base portions form a channel for receiving an end portion of a blood vessel.

According to another aspect of the present invention, a method is provided for repairing at least a portion of a blood vessel. One step of the method includes providing a vascular graft comprising an elongated main body portion having a first distal end portion defining a first opening and a second proximal end portion defining a second opening, a first sewing ring having a first base portion securely attached to the first distal end portion, and a second sewing ring having a second base portion securely attached to the first distal end portion at a location proximal to the first sewing ring, the first and second base portions forming a channel for receiving an end portion of a blood vessel. A placement position for the vascular graft is then determined at the portion of the blood vessel to be repaired. Next, the vascular graft is delivered to the portion of the blood vessel to be repaired, and the end portion of the blood vessel is positioned in the channel formed by the first and second sewing rings. The end portion of the blood vessel is then secured between the first and second sewing rings so that the lumen of the blood vessel to be repaired and the lumen of the elongated main body portion are in fluid communication with one another.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a plan view showing a vascular graft constructed in accordance with the present invention;

FIG. 2 is a perspective view showing first and second sewing rings of the vascular graft in FIG. 1;

FIG. 3 is an exploded perspective view showing a first distal end portion of the vascular graft in FIG. 1;

FIG. 4 is a plan view taken along Line 4-4 in FIG. 3;

FIG. 5A is a cross-sectional view taken along Line 5A-5A in FIG. 3

FIG. 5B is a cross-sectional view showing an alternative embodiment of the vascular graft in FIG. 5A;

FIG. 5C is a cross-sectional view showing another alternative embodiment of the vascular graft in FIG. 5A;

FIG. 6A is a plan view showing an alternative embodiment of the vascular graft in FIG. 1;

FIG. 6B is a cross-sectional view taken along Line 6B-6B in FIG. 6A;

FIG. 6C is a cross-sectional view showing an alternative embodiment of the vascular graft in FIG. 6B;

FIG. 7 is a perspective view of an abdominal aorta having a diseased portion (shaded region);

FIG. 8A is a perspective view showing an alternative embodiment of the vascular graft in FIG. 1 having an exploded configuration;

FIG. 8B is a perspective view showing the vascular graft in FIG. 8A having an assembled configuration;

FIG. 9 is a perspective view of the abdominal aorta in FIG. 7 with the diseased portion resected;

FIG. 10 is a perspective view showing the vascular graft of FIG. 8B being delivered to the abdominal aorta in a delivery sheath;

FIG. 11 is a perspective view showing the vascular graft in FIG. 10 upon removal from the delivery sheath;

FIG. 12 is a perspective view showing a second proximal end portion of the vascular graft in FIG. 11 in contact with an end portion of the abdominal aorta;

FIG. 13A is a cross-sectional view taken along Line 13A-13A in FIG. 12;

FIG. 13B is a cross-sectional view showing the second proximal end portion of the vascular graft in FIG. 12 anastomosed with the end portion of the abdominal aorta;

FIG. 14 is a perspective view showing a first distal end portion and the second proximal end portion of the vascular graft in FIG. 12 anastomosed with a portion of the abdominal aorta;

FIG. 15 is a perspective view showing the vascular graft of FIG. 14 implanted in the abdominal aorta;

FIG. 16A is a perspective view showing another alternative embodiment of the vascular graft in FIG. 1 having an exploded configuration;

FIG. 16B is a perspective view of the vascular graft in FIG. 16A having an assembled configuration;

FIG. 17 is a perspective view showing the vascular graft of FIG. 16B being delivered to an aortic arch aneurysm;

FIG. 18 is a perspective view showing a second elongated main body portion of the vascular graft in FIG. 17 expanded in the proximal descending aorta;

FIG. 19 is a perspective view showing an elongated main body portion of the vascular graft in FIG. 18 expanded in the aortic arch;

FIG. 20 is a perspective view showing the vascular graft of FIG. 19 implanted in the aortic arch aneurysm; and

FIG. 21 is a perspective view showing an alternative embodiment of the vascular graft in FIGS. 16A-B.

### DETAILED DESCRIPTION

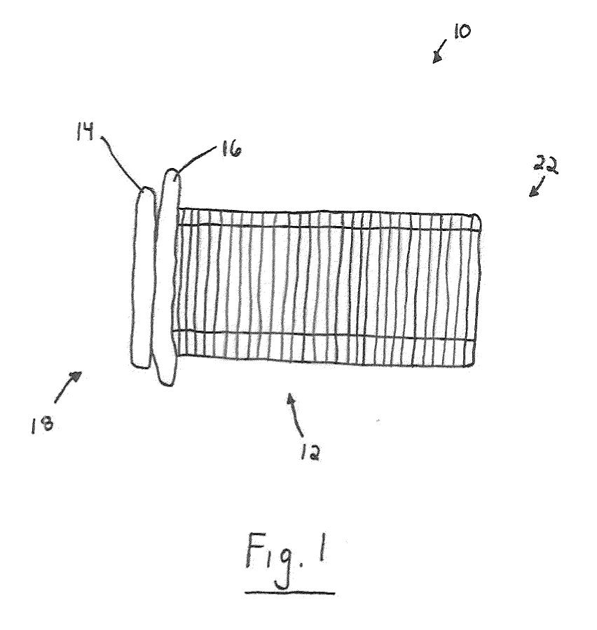

The present invention relates generally to surgical implants, and more particularly to a vascular graft adapted for anastomosis with at least one blood vessel. As representative of the present invention, FIG. 1 illustrates a vascular graft 10 for the treatment of vascular abnormalities, such as aortic and/or abdominal aneurysms. Additionally, the vascular graft 10 may be used to treat vascular trauma, atherosclerosis, arteriosclerosis, calcification, microbial infection, congenital defects, and other obstructive diseases associated with the aorta. Accordingly, the term “aortic aneurysm” as used herein is intended to relate to and include thoracic aneurysms, abdominal aneurysms, and related vessel diseases.

FIGS. 1-6C illustrate one aspect of the present invention. In FIG. 1, a vascular graft 10 comprises an elongated main body portion 12, a first sewing ring 14, and a second sewing ring 16. The vascular graft 10 has a generally flexible, tube-like configuration and is adapted for placement in or adjacent a bodily vessel, such as an artery or vein. The vascular graft 10 is configured to engage a bodily vessel so that a substantial seal is formed between the bodily vessel and the vascular graft. As described in more detail below, the vascular graft 10 can be compressed to facilitate delivery to a bodily vessel in need of repair, and then selectively expanded by, for example, a balloon, stent, etc. so that the vascular graft substantially conforms to the inner surface of the bodily vessel.

The vascular graft 10 can be comprised of any biocompatible material that is mechanically stable in vivo and is capable of preventing or substantially reducing the possibility of the passage or flow of blood (or other body fluids) through the vascular graft. Examples of suitable materials for use in constructing the vascular graft 10 can include biocompatible plastics, such as woven polyester, non-resorbable elastomers or polymers such as silicone, SBR, EPDM, butyl, polyisoprene, Nitril, Neoprene, nylon alloys and blends, poly(ethylene-vinyl-acetate) (EVA) copolymers, silicone rubber, polyamides, polyurethane, poly(ester urethanes), poly(ether urethanes), poly(ester urea), polypropylene, polyethylene, polycarbonate, polytetrafluoroethylene (PTFE) (e.g., TEFLON), expanded PTFE (ePTFE), polyethylene terephthalate (e.g., DACRON), and polyethylene copolymers.

The vascular graft 10 can also include a layer of biological material (not shown), such as bovine or equine pericardium, peritoneal tissue, an allograft, a homograft, a patient graft, or a cell-seeded tissue. The layer of biological material can cover the entire vascular graft 10 or only a portion thereof. One skilled in the art will appreciate that other materials suitable for vascular surgical applications may also be appropriate for the vascular graft 10.

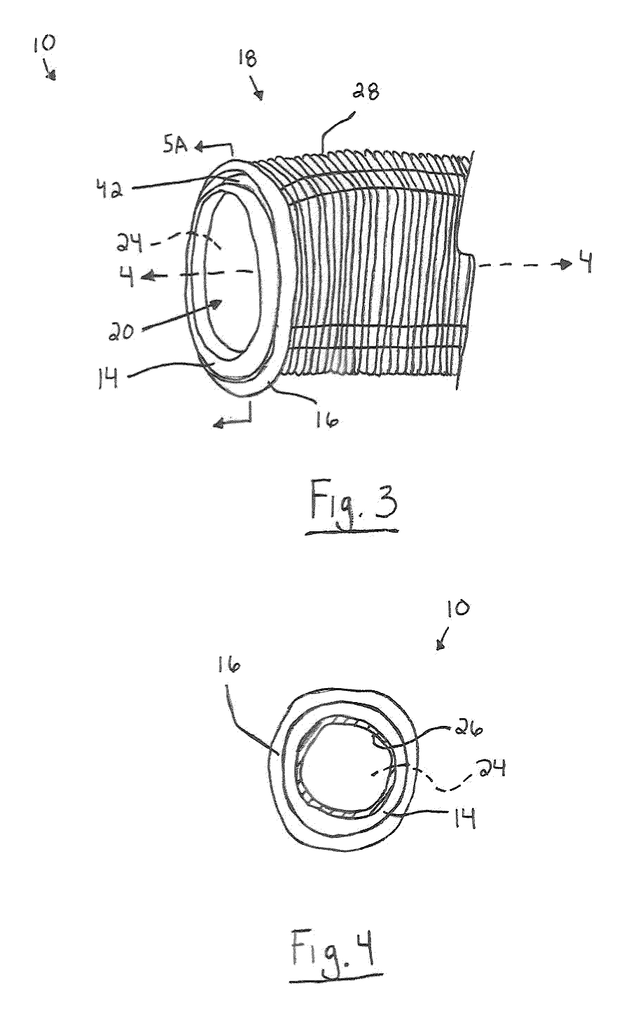

As shown in FIG. 1, the vascular graft 10 comprises an elongated main body portion 12 having a first distal end portion 18 defining a first opening 20 (FIG. 3) and a second proximal end portion 22 defining a second opening (not shown). The elongated main body portion 12 includes a lumen 24 extending between the first distal end portion 18 and the second proximal end portion 22. The lumen 24 is defined by an inner surface 26 (FIG. 4) and an outer surface 28 (FIG. 3). The elongated main body portion 12 can have any shape and size to facilitate surgical placement of the vascular graft 10 so that the elongated main body portion conforms or substantially conforms to the inner surface of a bodily vessel.

It will be appreciated that the elongated main body portion 12 can also have any shape and size to facilitate partial or complete replacement or repair of a bodily vessel. Additionally, it should be appreciated that the elongated main body portion 12 can have a configuration other than those illustrated in FIGS. 1-16.

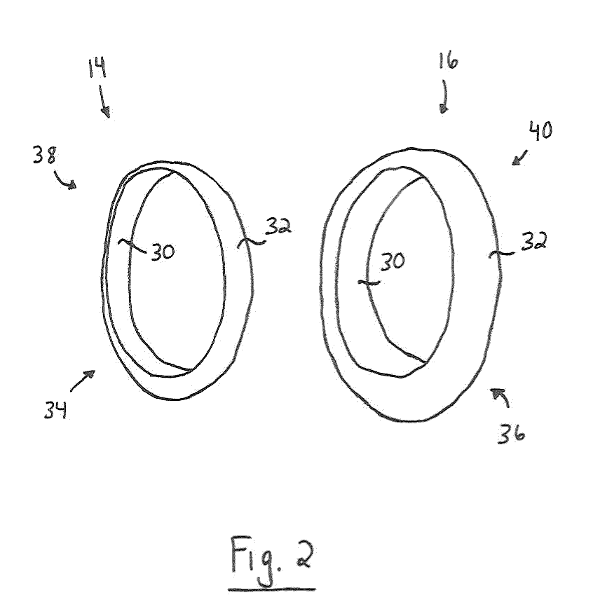

The first distal end portion 18 of the elongated main body portion 12 includes first and second sewing rings 14 and 16 securely attached thereto (but not integrally formed with). Each of the first and second sewing rings 14 and 16 (FIG. 2) has an annular shape and includes a thickness T1 and T2 defined by an inner surface 30 and an outer surface 32. As shown in FIG. 2, the thickness T2 of the second sewing ring 16 is greater than the thickness T1 of the first sewing ring 14; however, it will be appreciated that the thickness T1 of the first sewing ring can be substantially equal to the thickness T2 of the second sewing ring. Each of the first and second sewing rings 14 and 16 also respectively comprises first and second base portions 34 and 36 integrally formed with first and second upper portions 38 and 40. As described below, the first and second base portions 34 and 36 are adapted for attachment to the elongated main body portion 12 of the vascular graft 10. Although not shown in FIG. 1, it will be appreciated that the second proximal end portion 22 of the vascular graft 10 can also include first and second sewing rings 14 and 16.

The first and second sewing rings 14 and 16 can be made of any one or combination of biocompatible materials including, for example, a synthetic fiber, such as PTFE, or a polyester (e.g., DACRON) mesh weave. The material(s) used to make the first and second sewing rings 14 and 16 can also include interstices (not shown) permeable to tissue in-growth. The material may be filled with a biologically acceptable, spongy material, such as silicone rubber, polyurethane, or a hydrogel to facilitate formation or shaping of the first and second sewing rings 14 and 16. Although the first and second sewing rings 14 and 16 generally have a circular cross-sectional profile, it will be appreciated that the sewing rings can have any desired cross-sectional profile (e.g., ovoid or rectangular). It will also be appreciated that the first and second sewing rings 14 and 16 can be attached to the elongated main body portion 12 using sutures, for example, or any other suitable attachment means, such as staples or clips.

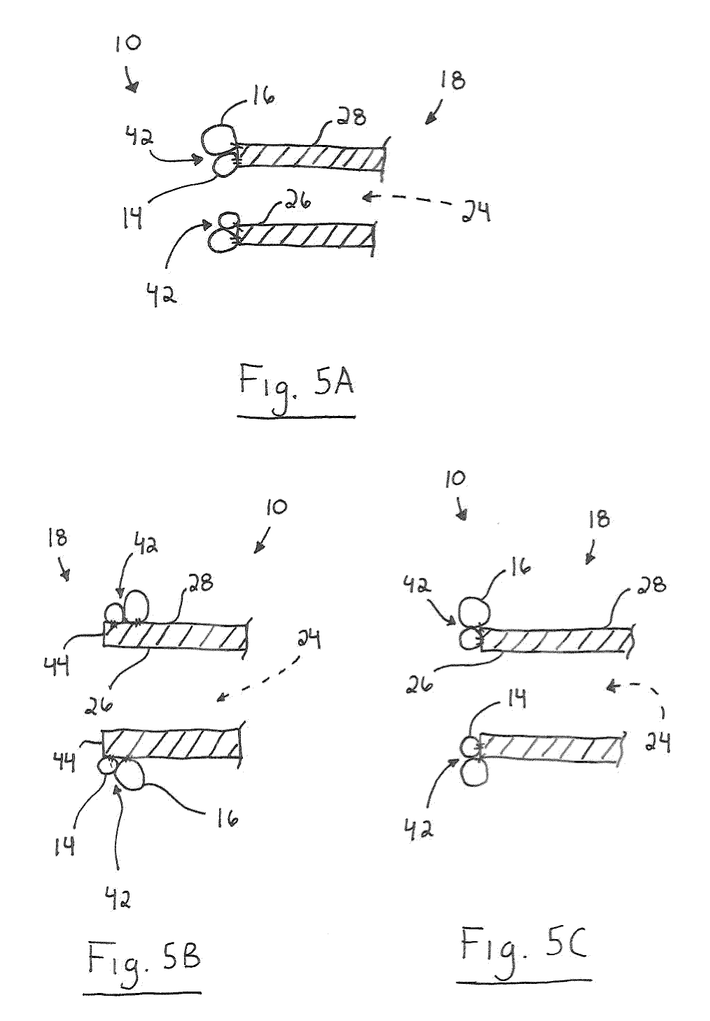

As shown in FIG. 5A, the first and second base portions 34 and 36 of the first and second sewing rings 14 and 16, respectively, are securely attached to the elongated main body portion 12 so that a channel 42 capable of receiving an end portion of a blood vessel is formed between the first and second upper portions 38 and 40. It will be appreciated that the channel 42 can be formed between the first and second base portions 34 and 36 or a combination of the first and second base portions and the first and second upper portions 40 and 42, depending upon the size and thickness of the blood vessel and the thickness T1 and T2 of the first and second sewing rings 14 and 16.

The first and second base portions 34 and 36 are securely attached to a distal end 44 of the elongated main body portion 12 to form the channel 42 therebetween (FIG. 5A). Alternatively, the first and second base portions 34 and 36 can be securely attached to the outer surface 28 of the elongated main body portion 12 so that the base portions are in contact with one another (FIG. 5B). It will be appreciated, however, that first base portion 34 can be securely attached to a distal end 44 of the elongated main body portion 12 and the second base portion 36 securely attached to the outer surface 28 of the elongated main body portion (FIG. 5C).

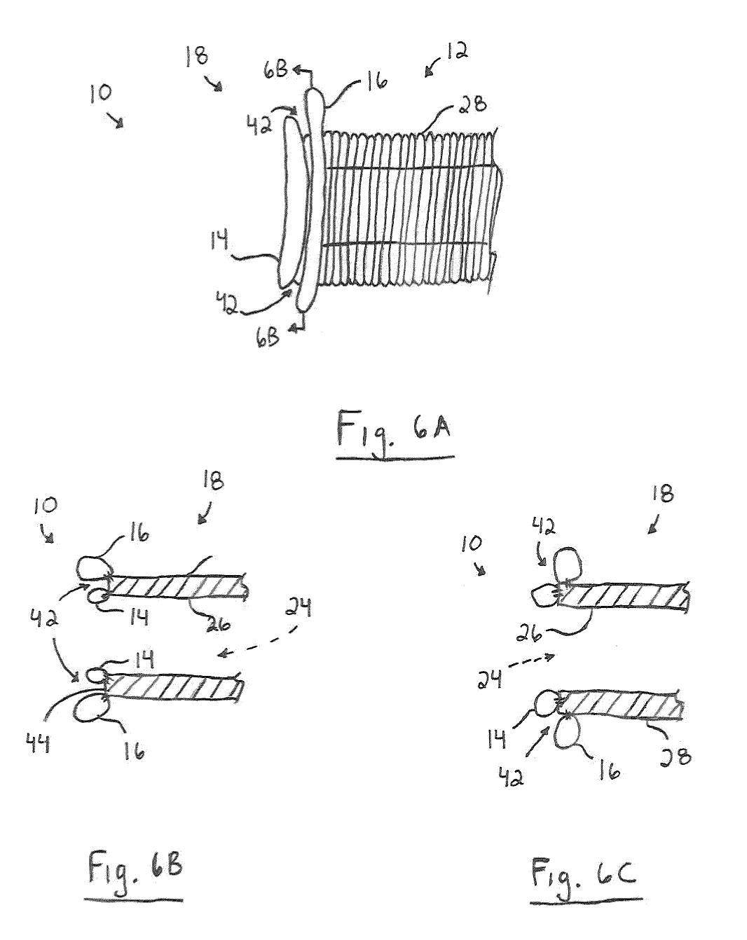

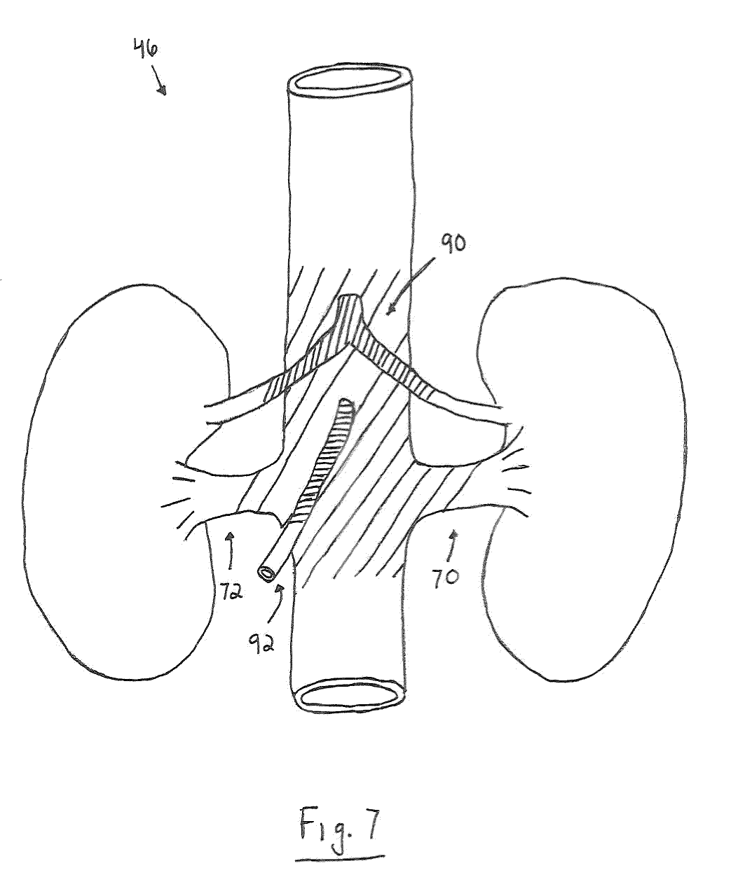

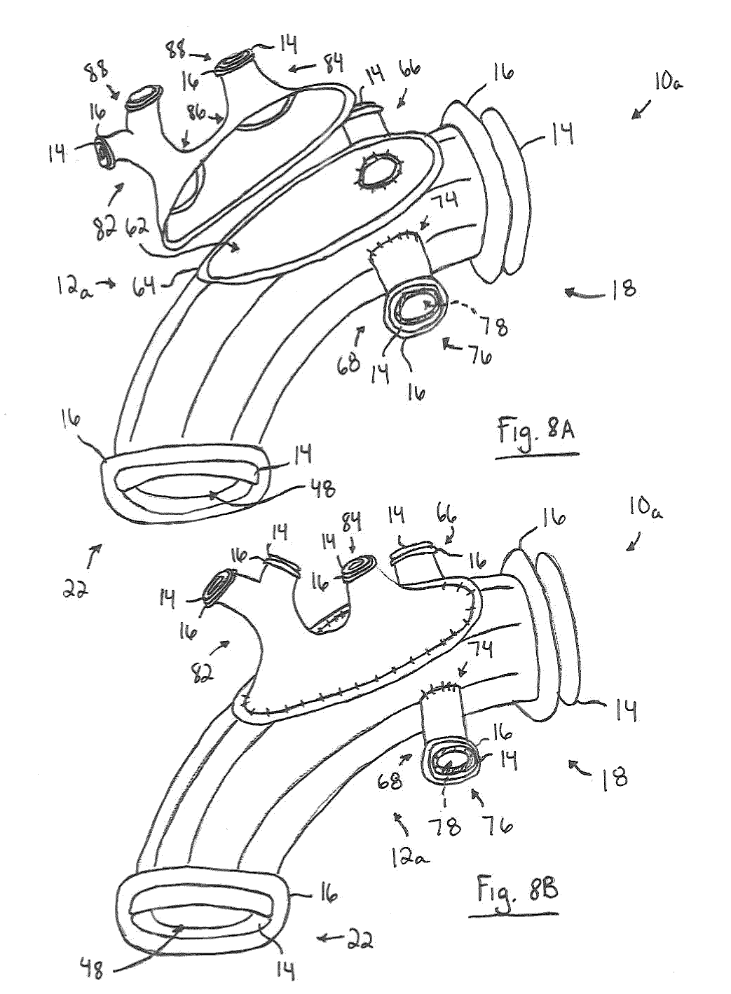

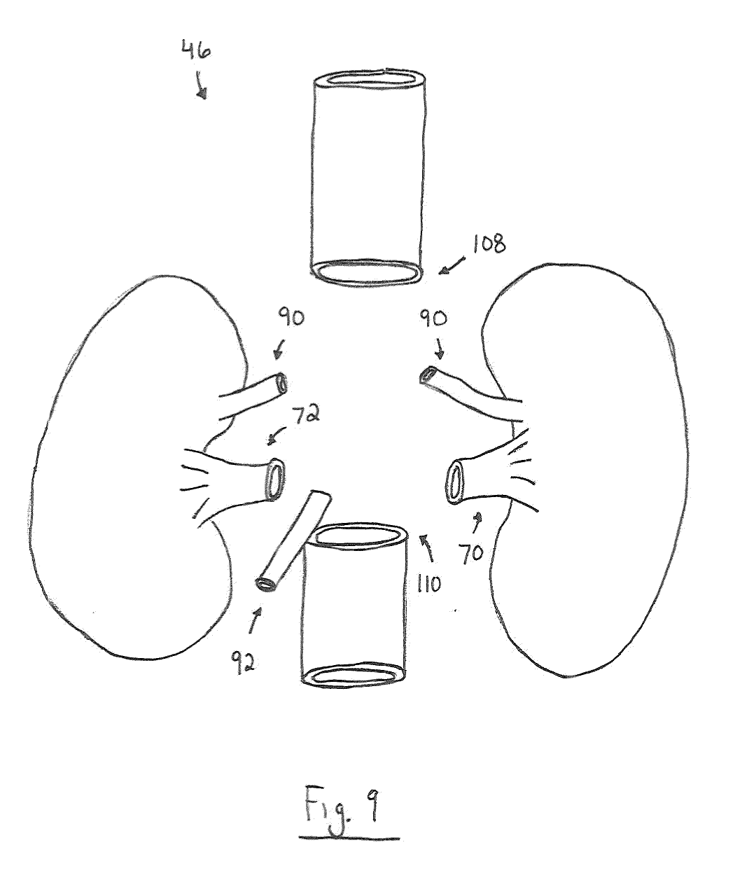

Referring to FIGS. 6A-C, it will be appreciated that the channel 42 may alternatively be formed between the first and second base portions 34 and 36 by spacing apart the first and second sewing rings 14 and 16. As shown in FIG. 6B, for example, the first and second base portions 34 and 36 can be spaced apart from one another and securely attached to the distal end 44 of the elongated main body portion 12. Alternatively, the first and second base portions 34 and 36 can be securely attached to the distal end 44 and the outer surface 28 of the elongated main body portion 12, respectively (FIG. 6C). Although not shown in FIGS. 6A-C, it will additionally be appreciated that the first and second base portions 34 and 36 can be spaced apart from one another and securely attached to the outer surface 28 of the elongated main body portion 12.

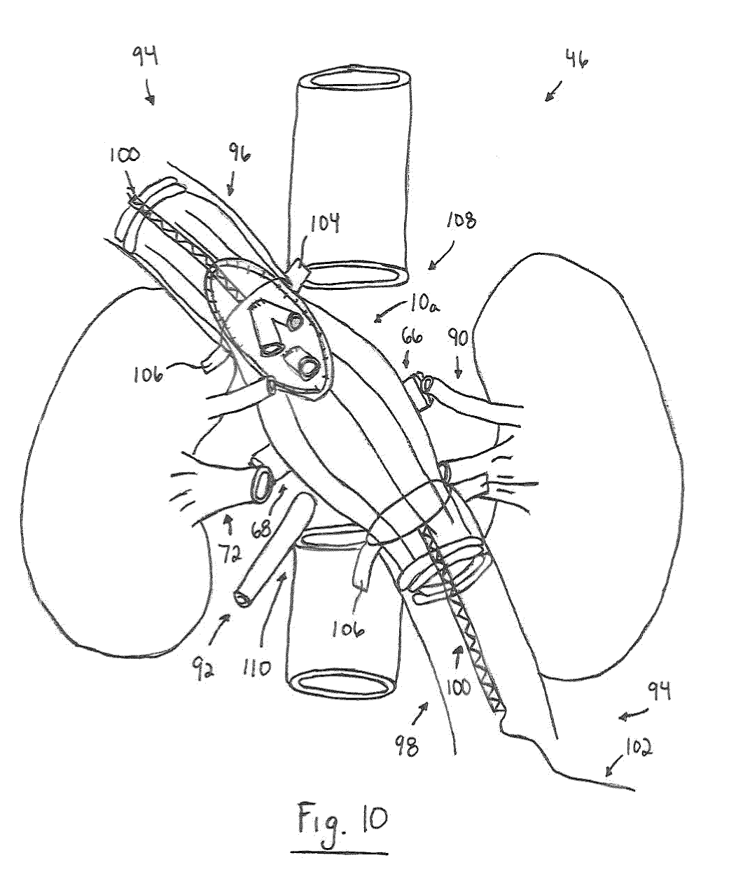

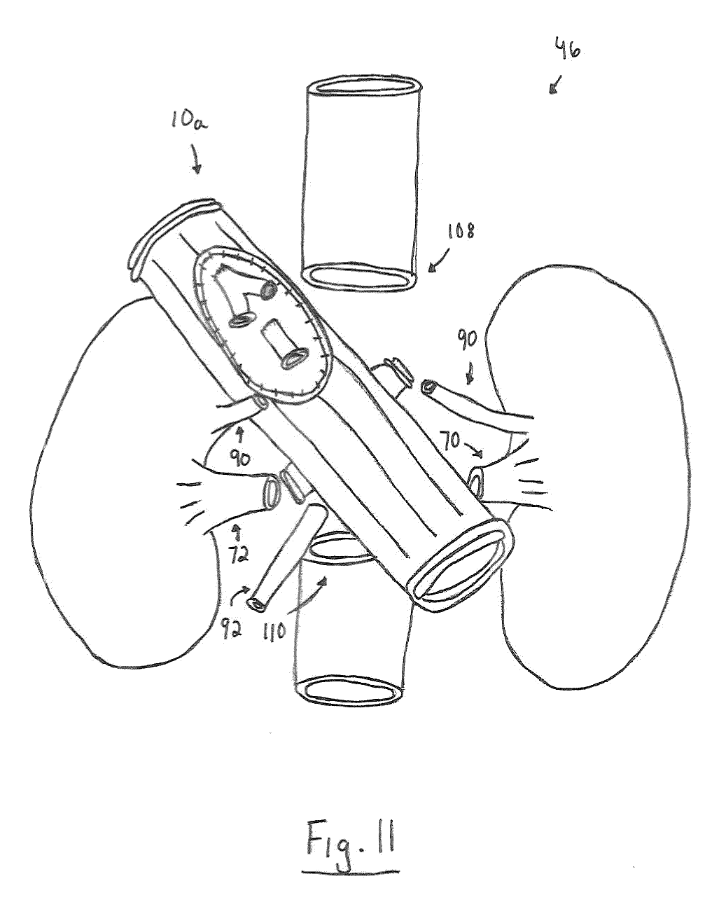

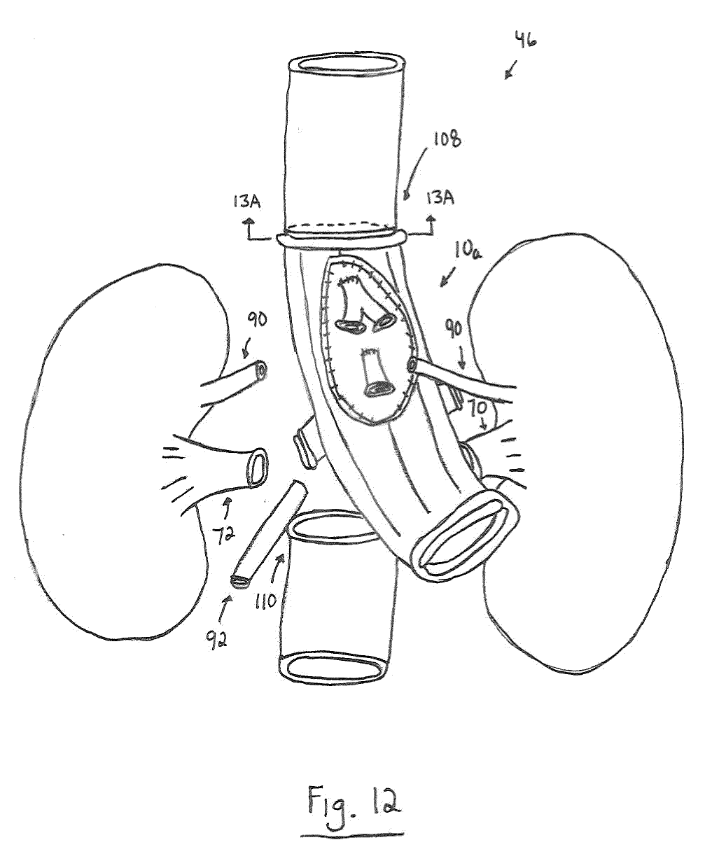

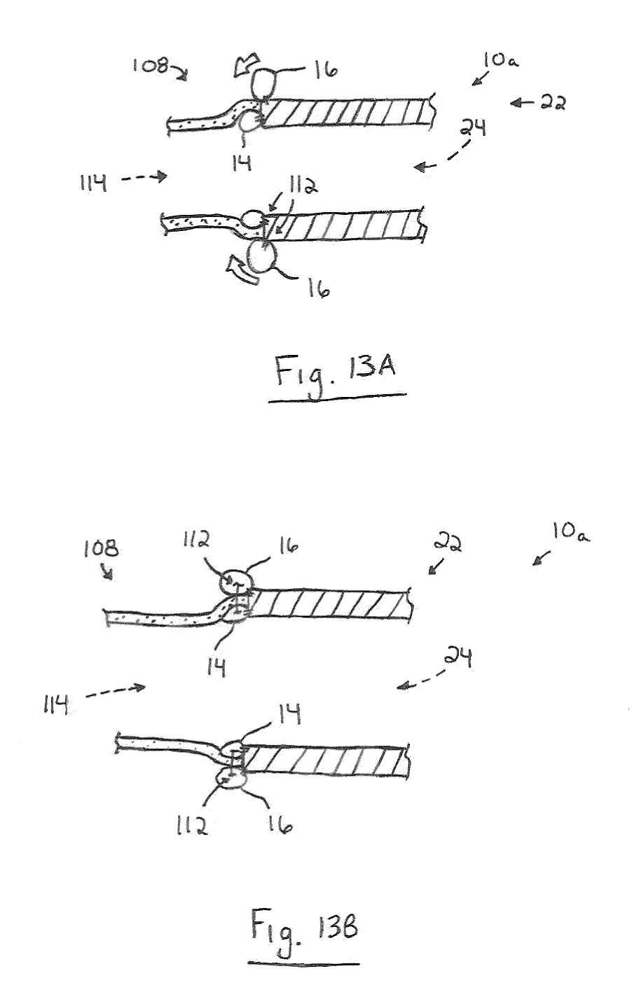

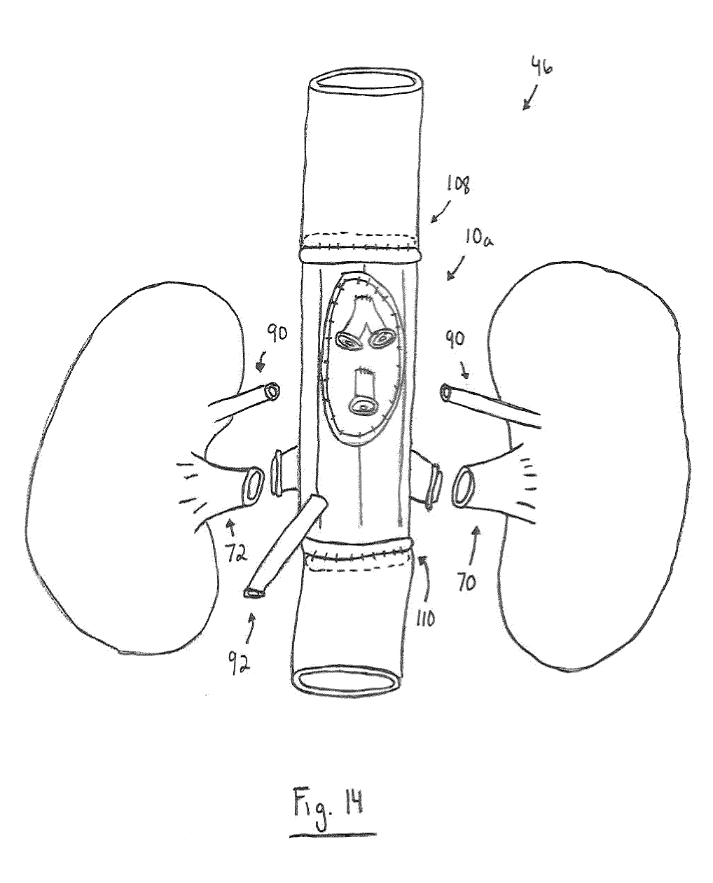

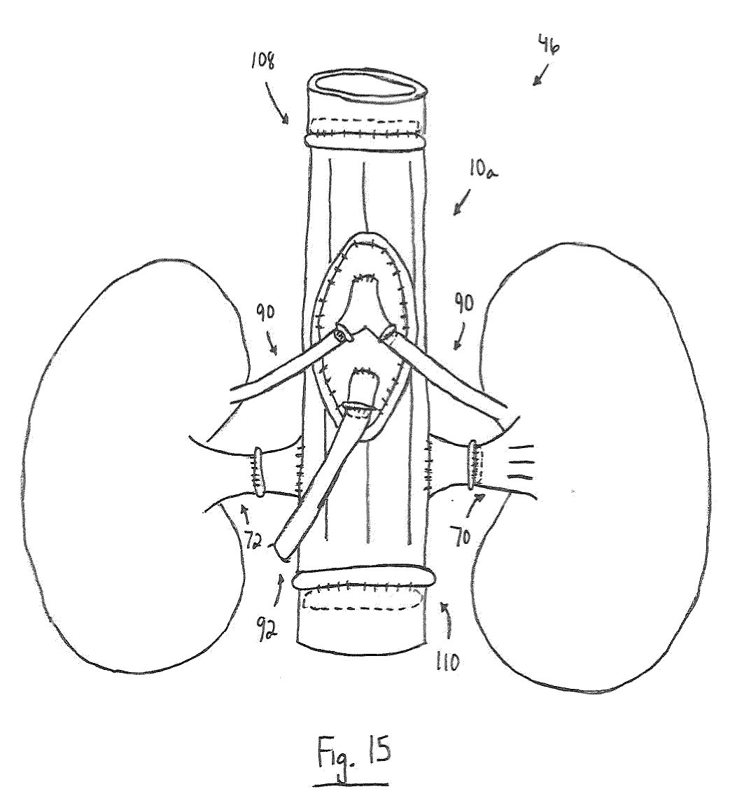

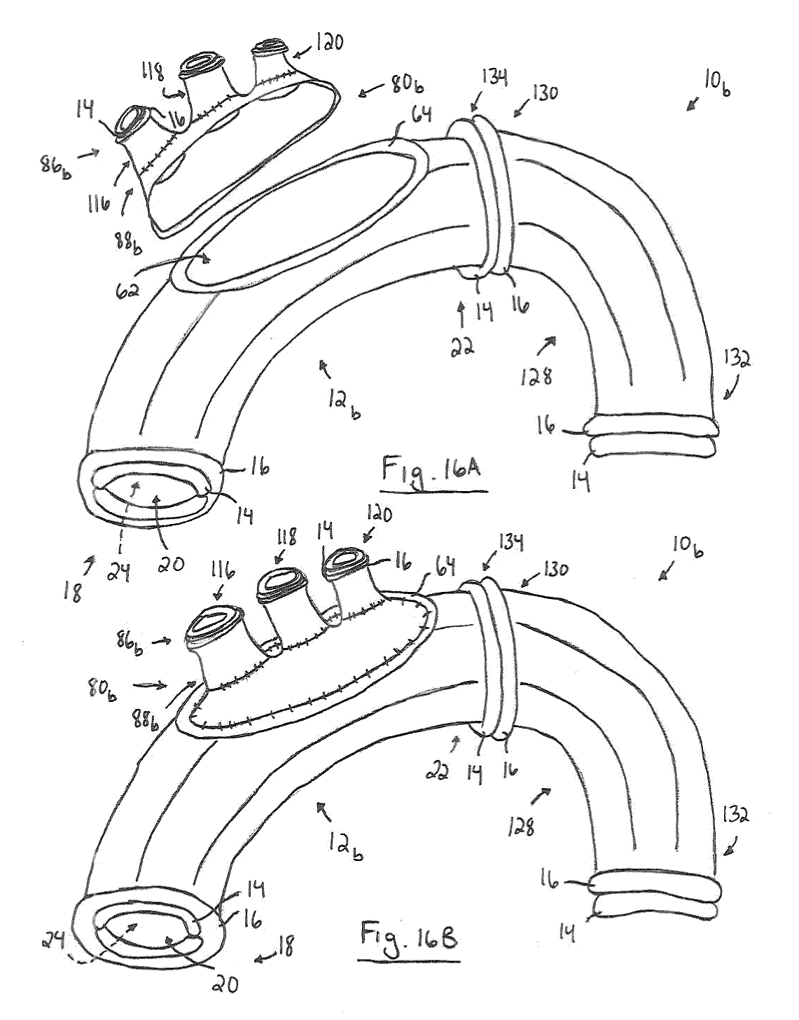

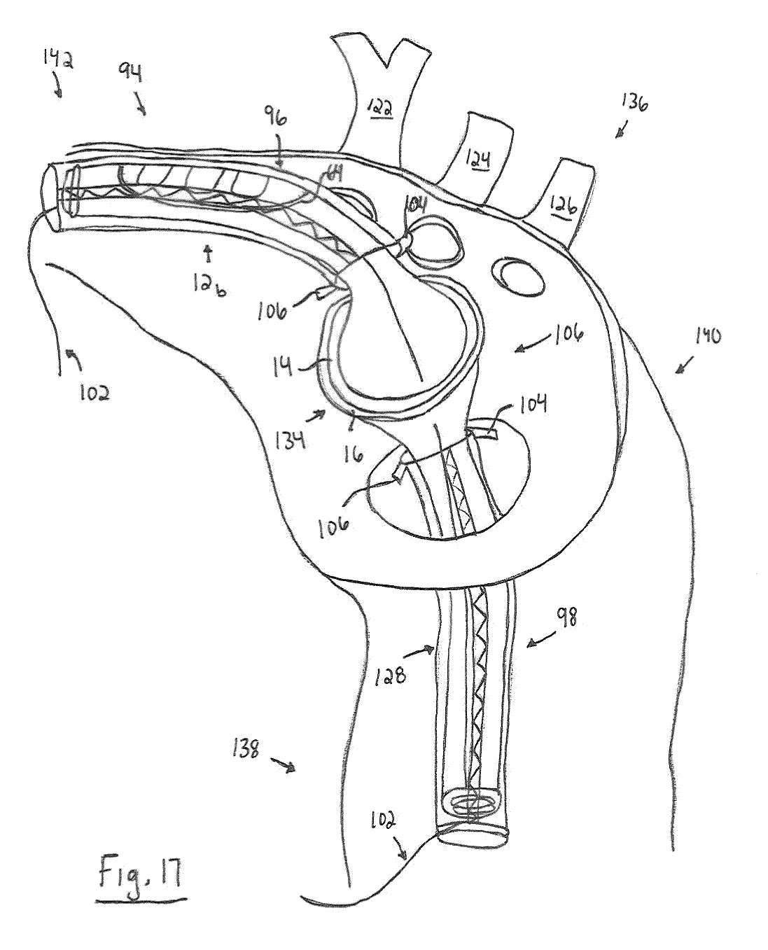

FIGS. 7-15 illustrate another aspect of the present invention comprising a method for repairing at least a portion of a blood vessel, such as an abdominal aorta 46 (FIG. 7). As indicated by the shaded region in FIG. 8, the portion of the abdominal aorta 46 to be repaired can include an abdominal aortic aneurysm (AAA). AAA is a localized dilatation of the abdominal aorta 46 that exceeds the normal diameter. AAA is caused by a degenerative process of the aortic wall whose exact etiology remains unknown. AAA is most commonly located infrarenally; however, other locations, such as suprarenally, pararenally, thoraco-abdominally, or in the thoracic aorta are also possible.

One step of the method comprises providing a vascular graft 10a. FIGS. 8A-B illustrate one example of a vascular graft 10a that may be used to repair the AAA. The vascular graft 10a shown in FIGS. 7-15 is identically constructed as the vascular graft 10 shown in FIGS. 1-6C, except as described below. In FIGS. 7-15, structures that are identical as structures in FIGS. 1-6C use the same reference numbers, whereas structures that are similar but not identical carry the suffix “a”.

As shown in FIGS. 8A-B, the vascular graft 10a comprises an elongated main body portion 12a having a first distal end portion 18 defining a first opening (not shown) and a second proximal end portion 22 defining a second opening 48. Each of the second proximal end portion 22 and the first distal end portion 18 includes first and second sewing rings 14 and 16. The elongated main body portion 12a includes a lumen 24 extending between the first distal end portion 18 and the second distal end portion 22. The elongated main body portion 12a also includes an aperture 62 with an attachment ring 64 securely attached thereto, and first and second arm members 66 and 68 respectively configured to accommodate the left and right renal arteries 70 and 72 (FIG. 7).

The first and second arm members 66 and 68 (FIGS. 8A-B) have a generally tube-like configuration and include first and second ends 74 and 76. The first and second arm members 66 and 68 also include a lumen 78 extending between the first and second ends 74 and 76. The first end 74 of each of the first and second arm members 66 and 68 is securely attached to the elongated main body portion 12a so that the lumen 78 of each of the arm members is in fluid communication with the lumen 24 of the elongated main body portion.

The second end 76 of each of the first and second arm members 66 and 68 is respectively configured to accommodate the left and right renal arteries 70 and 72 (FIG. 7). As shown in FIGS. 8A-B, the second end 76 of each of the first and second arm members 66 and 68 includes first and second sewing rings 14 and 16. The first and second arm members 66 and 68 are securely attached to the elongated main body portion 12a using sutures, for example. The first and second arm members 66 and 68 can be made of a biocompatible material, such as woven polyester, DACRON, PTFE and/or TEFLON.

As shown in FIG. 8B, the vascular graft 10a also includes a multi-lumen branch graft 80 comprising first and second branch members 82 and 84 securely attached to the attachment ring 64. Each of the first and second branch members 82 and 84 has a tubular configuration and includes first and second ends 86 and 88. The second end 88 of the first branch member 82 has a bifurcated configuration to accommodate a celiac trunk 90 (FIG. 7), and the second end (FIGS. 8A-B) of the second branch member 84 is configured to accommodate a superior mesenteric artery 92 (FIG. 7). The second end 88 of each of the first and second branch members 82 and 84 includes first and second sewing rings 14 and 16 securely attached thereto. The first and second branch members 82 and 84 can be made of a biocompatible material, such as woven polyester, DACRON, PTFE and/or TEFLON.

It should be appreciated that the attachment ring 64 can have a configuration identical or similar to the configuration of the first and second sewing rings 14 and 16. This configuration of the attachment ring 64 may be useful where the multi-lumen branch graft 80 is not included as part of the vascular graft 10a and, instead, a portion of the native abdominal aorta 46 which includes the celiac trunk 90 and the superior mesenteric artery 92 can be securely anastomosed with the attachment ring.

A placement position is determined for the vascular graft 10a at the portion of the abdominal aorta 46 to be repaired. To determine the placement position, one or a combination of known imaging techniques, such as ultrasonography, fluoroscopy, angiography, CT, helical CT, CT angiogram, MRI, and/or MR angiography is used. After identifying the placement position, the subject is prepared for surgery. Although implantation of the vascular graft 10a is described below using an open-abdominal surgical approach, it will be appreciated that other methods for implanting the vascular graft, such as a percutaneous or minimally invasive surgical technique may also be used.

Prior to implantation of the vascular graft 10a, the vascular graft is loaded into a delivery sheath 94 (FIG. 10) to facilitate delivery of the vascular graft. The delivery sheath 94 maintains the vascular graft 10a in a sterile environment while also keeping the vascular graft in a compressed configuration prior to implantation. Although not shown in detail, the delivery sheath 94 comprises first and second envelope members 96 and 98 capable of containing respective portions of the vascular graft 10a in a compressed configuration. Each of the first and second envelope members 96 and 98 includes a release mechanism 100 (or any other suitable alternative) for selectively releasing the vascular graft 10a from the delivery sheath 94.

As shown in FIG. 10, the release mechanism 100 includes at least one string 102 or line which, when pulled or retracted, causes the release mechanism to separate each of the first and second envelope members 96 and 98 and thereby release the respective portions of the vascular graft 10a from the envelope members. The delivery sheath 94 can be made of a transparent, biocompatible material (e.g., a plastic polymer) to facilitate visualization of the vascular graft 10a during implantation. It will be appreciated that the release mechanism 100 can also include first and second tab members 104 and 106. The first and second tab members 104 and 106 can be manipulated by, for example, tactile means to progressively peel away the delivery sheath 94 and thereby deliver the vascular graft 10a to the placement position.

After loading the vascular graft 10a into the delivery sheath 94, an incision (not shown) is made over the skin of the subject and through the muscle (not shown) overlying the abdominal aorta 46. The abdominal tissue (not shown) surrounding the abdominal aorta 46 is then manipulated to clearly expose the AAA. Next, the blood vessels superior and inferior to the AAA are tied off or clamped (not shown) to temporarily stop blood flow through the abdominal aorta 46. For example, portions of the abdominal aorta 46 both superior and inferior to the AAA are clamped. Additionally, portions of the left and right renal arteries 70 and 72, as well as the celiac trunk 90 and superior mesenteric artery 92 are clamped to temporarily prevent blood flow through the AAA. After clamping the vessels surrounding AAA, the diseased portion of the abdominal aorta 46 is resected as shown in FIG. 9.

The delivery sheath 94 containing the vascular graft 10a is next positioned over the abdominal aorta 46 (FIG. 10). More particularly, the second proximal end portion 22 of the vascular graft 10a is positioned adjacent the end portion 108 of the abdominal aorta 46 superior to the left and right renal arteries 70 and 72. Additionally, the first distal end portion 18 of the vascular graft 10a is positioned adjacent the end portion 110 of the abdominal aorta 46 inferior to the left and right renal arteries 70 and 72. Next, the string 102 of each of the first and second envelope members 96 and 98 is manipulated (e.g., pulled) so that the first and second envelope members begin to release the vascular graft 10a. As the first and second envelope members 96 and 98 release the vascular graft 10a, the elongated main body portion 12a begins to expand into the AAA (FIG. 11).

Upon completely removing the vascular graft 10a from the delivery sheath 94, the end portion 108 of the abdominal aorta 46 superior to the left and right renal arteries 70 and 72 is positioned in the channel 42 formed by the first and second sewing rings 14 and 16 (FIG. 13A). As indicated by the arrows in FIG. 13A, the second upper portion 40 of the second sewing ring 16 is then folded over the end portion 108 of the abdominal aorta 46 to sandwich the end portion between the first upper portion 38 and the second upper portion of the first sewing ring 14 and the second sewing ring, respectively. At least one suture 112 is passed through the first sewing ring 14, the end portion 108 of the abdominal aorta 46 (located in the channel 42), and the second sewing ring 16 in a circumferential manner so that the lumen 24 of the elongated main body portion 12a and the lumen 114 of the abdominal aorta are in fluid communication with one another (FIG. 13B). This provides a double hemostatic effect.

After securely anastomosing the end portion 108 of the abdominal aorta 46 superior to the left and right renal arteries 70 and 72 with the second proximal end portion 22, the end portion 110 of the abdominal aorta inferior to the renal arteries is anastomosed with the first distal end portion 18 of the vascular graft 10a in a manner substantially identical to the one described above (i.e., for anastomosing the end portion of the abdominal aorta superior to the renal arteries with the second proximal end portion of the vascular graft). As shown in FIG. 15, the left and right renal arteries 70 and 72 are then respectively anastomosed (as described above) with the first and second arm members 66 and 68 of the vascular graft 10a, and the celiac trunk 90 and the superior mesenteric artery 92 are respectively anastomosed with the first and second branch members 82 and 84 (as described above). After the vascular graft 10a is securely positioned in the abdominal aorta 46, the clamps are removed and normal blood flow can resume through the abdominal aorta. To complete the surgery, the abdominal tissue is returned to its place over the abdominal aorta 46 and the incision is closed with sutures (not shown).

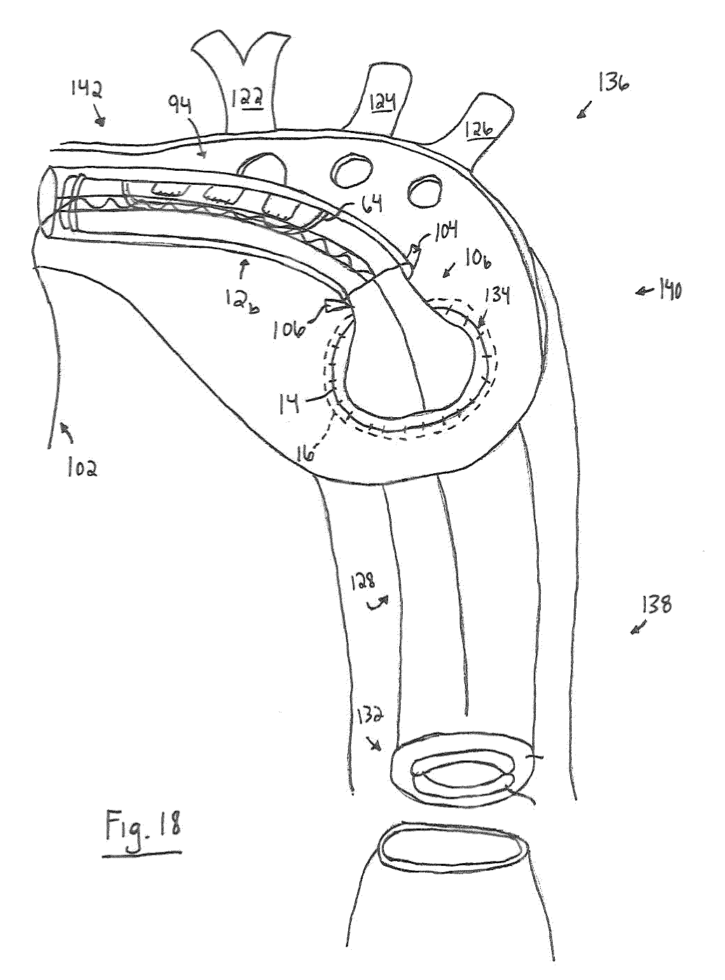

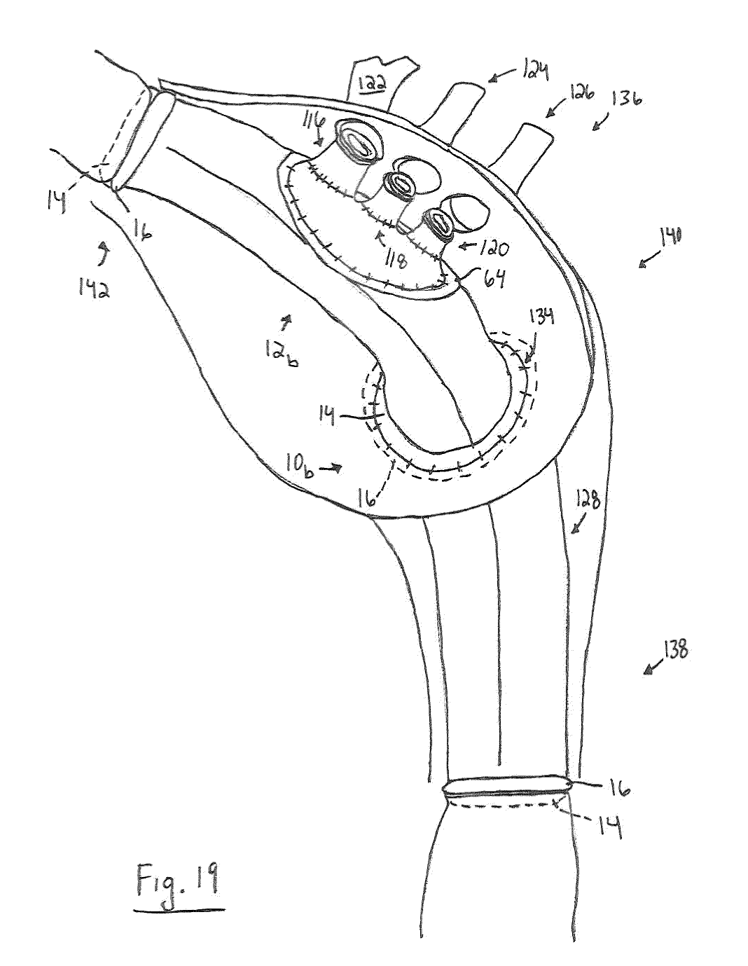

Another aspect of the present invention is illustrated in FIGS. 16-20. In FIGS. 16-20, a method is provided for repairing at least a portion of a blood vessel, such as an aortic arch aneurysm (FIG. 17). To repair an aortic arch aneurysm, a vascular graft 10b, such as the one illustrated in FIGS. 16A-B can be used. The vascular graft 10b shown in FIGS. 16A-B can be identically constructed as the vascular graft 10 shown in FIGS. 1-6C, except as described below. In FIGS. 16-20, structures that are identical as structures in FIGS. 1-6C use the same reference numbers, whereas structures that are similar but not identical carry the suffix “b”.

One step of the method can include providing the vascular graft 10b shown in FIGS. 16A-B. The vascular graft 10b can comprise an elongated main body portion 12b having a first distal end portion 18 defining a first opening 20, and a second proximal end portion 22 defining a second opening (not shown). Additionally, the elongated main body portion 12b can also include an aperture 62 with an attachment ring 64 securely attached thereto. The elongated main body portion 12b can also include first and second sewing rings 14 and 16 securely attached to the first distal end portion 18, and a multi-lumen branch graft 80b adapted for anastomosis with the aperture 62.

It should be appreciated that the attachment ring 64 can have a configuration identical or similar to the configuration of the first and second sewing rings 14 and 16 (FIG. 21). This configuration of the attachment ring 64 may be useful where the multi-lumen branch graft 80 is not included as part of the vascular graft 10b and, instead, a portion of the native aortic arch 136, which includes the brachiocephalic artery 122, the left common carotid artery 124, and the left subclavian artery 126 can be securely anastomosed with the attachment ring.

The multi-lumen branch graft 80b can include first, second, and third branch members 116, 118 and 120. Each of the first, second, and third branch members 116, 118, and 120 can have a generally tubular configuration and include first and second ends 86b and 88b. The second end 88b of each of the first, second, and third branch members 116, 118, and 120 can include first and second sewing rings 14 and 16. Additionally, the second end 88b of each of the first, second, and third branch members 116, 118, and 120 can be respectively configured to anastomose with or accommodate a brachiocephalic trunk artery 122 (FIG. 17), a left common carotid artery 124, and a left subclavian artery 126.

The vascular graft 10b (FIGS. 16A-B) can also include a second elongated main body portion 128 having a first end portion 130 defining a first opening (not shown) and a second end portion 132 defining a second opening (not shown). The second elongated main body portion 128 can have an elongated, tube-like configuration and include a second lumen (not shown) extending between the first and second end portions 130 and 132. As shown in FIGS. 16A-B, the second end portion 132 of the second elongated main body portion 128 can include first and second sewing rings 14 and 16.

The second elongated main body portion 128 can have any shape and size to facilitate placement of the vascular graft 10b so that the second elongated main body portion conforms or substantially conforms to the inner surface of a bodily vessel. The second elongated main body portion 128 can be made of a biocompatible material, such as woven polyester, DACRON, PTFE and/or TEFLON. The material used to construct the second elongated main body portion 128 can be the same or nearly the same as the material used to construct the elongated main body portion 12b.

The first end portion 130 of the second elongated main body portion 128 can be securely attached to the second proximal end portion 22 of the elongated main body portion 12b via a second attachment ring 134 comprising first and second sewing rings 14 and 16 or, alternatively, the elongated main body portion itself. The second elongated main body portion 128 can be securely attached to the elongated main body portion 12b so that the lumen of the second elongated main body portion is in fluid communication with the lumen 24 of the elongated main body portion. The second attachment ring 134 can enable repair of complex aortic lesions that involve both the aortic arch 136 and the descending aorta 138, even in the presence of a size mismatch between the vascular graft 10b and the aorta by covering the gap between the aorta and the vascular graft (e.g., during an elephant trunk procedure). The second elongated main body portion 128 can be securely attached to the second attachment ring 134 using sutures, for example, or any other known attachment means (e.g., staples, clips, adhesives, etc.).

To repair the aortic arch aneurysm, an open-chest elephant trunk procedure can be employed. Although implantation of the vascular graft 10b is described below using an open surgical approach, it will be appreciated that other methods for implanting the vascular graft, such as a percutaneous or minimally invasive surgical technique may also be used. After providing the vascular graft 10b shown in FIGS. 16A-B, a placement position for the vascular graft in the aortic arch aneurysm can be determined using a known imaging technique, such as fluoroscopy, angiography, ultrasonography, CT, helical CT, CT angiogram, MRI, and/or MR angiography.

Prior to implanting the vascular graft 10b, the vascular graft can be loaded into a delivery sheath 94 (FIG. 17). The delivery sheath 94 can facilitate delivery of the vascular graft 10b by maintaining the vascular graft in a sterile environment while also keeping the vascular graft in a compressed configuration prior to implantation. As shown in FIG. 17, the delivery sheath 94 can be constructed in an identical or similar manner as described above.

After loading the vascular graft 10b into the delivery sheath 94, the delivery sheath can be inserted into the aortic arch 136 via an incision (not shown). As shown in FIG. 17, the second elongated main body portion 128 of the vascular graft 10b can be positioned in the descending aorta 138, and the second attachment ring 134 can be positioned over a proximal portion 140 of the descending aorta. After positioning the second elongated main body portion 128 in the descending aorta 138, the string 102 of the second envelope member 98 can be manipulated (e.g., pulled) so that the second envelope member releases the second elongated main body portion and the second elongated main body portion expands into the descending aorta (FIG. 18).

Next, a portion of tissue comprising the aortic arch 136 and a portion of tissue comprising the descending aorta 138 can be positioned in the channel 42 formed between the first and second sewing rings 14 and 16 of the second attachment ring 134. As shown in FIG. 18, the portions of the aortic arch 136 and the descending aorta 138 can be anastomosed with the first and second sewing rings 14 and 16 of the second attachment ring 134 in a manner similar or identical as shown in FIGS. 13A-B (described above). Anastomosis of the portions of the aortic arch 136 and the descending aorta 138 with the first and second sewing rings 14 and 16 of the second attachment ring 134 provides a double hemostatic effect. It will be appreciated that other vascular structures can be anastomosed with the second attachment ring 134 as part of a second-stage elephant trunk procedure.

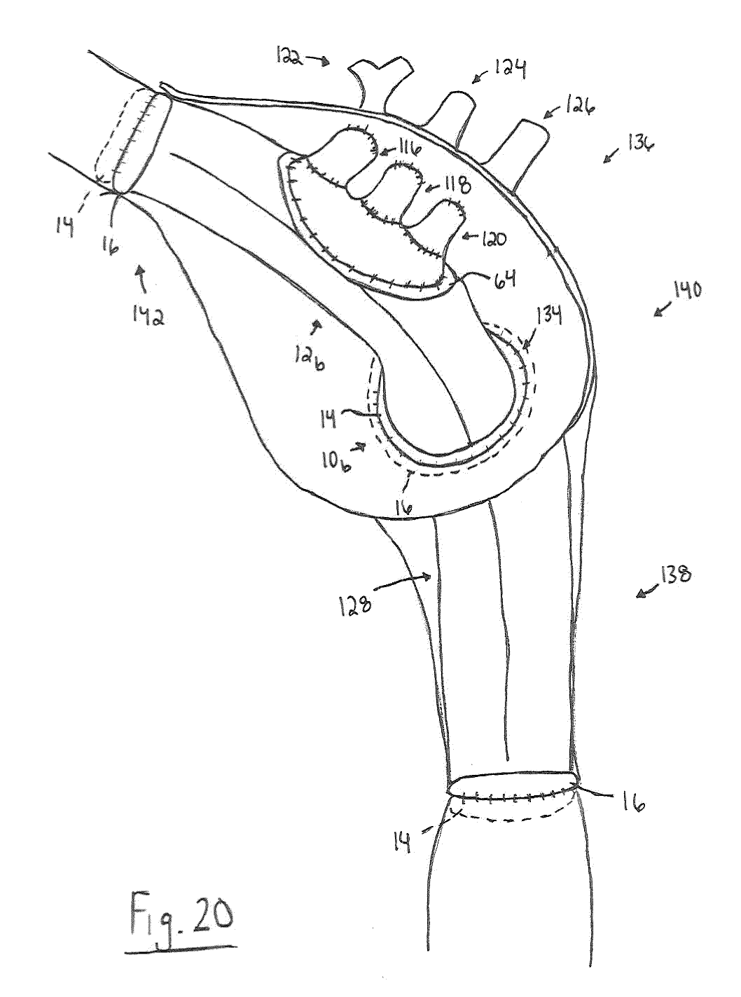

After securely attaching the second attachment ring 134 to the proximal portion 140 of the descending aorta 138, the string 102 of the first envelope member 96 can be manipulated (e.g., pulled) so that the first envelope member releases the elongated main body portion 12b and the elongated main body portion expands into the aortic arch 136 (FIG. 19). As shown in FIG. 19, a portion of the descending aorta 138 distal to the second end portion 132 of the second elongated main body portion 128 is moved into the channel 42 formed between the first and second sewing rings 14 and 16 at the second end portion. Additionally, a portion of the ascending aorta 142 proximal to the first distal end portion 18 of the elongated main body portion 12b is moved into the channel 42 formed between the first and second sewing rings 14 and 16 at the first distal end portion.

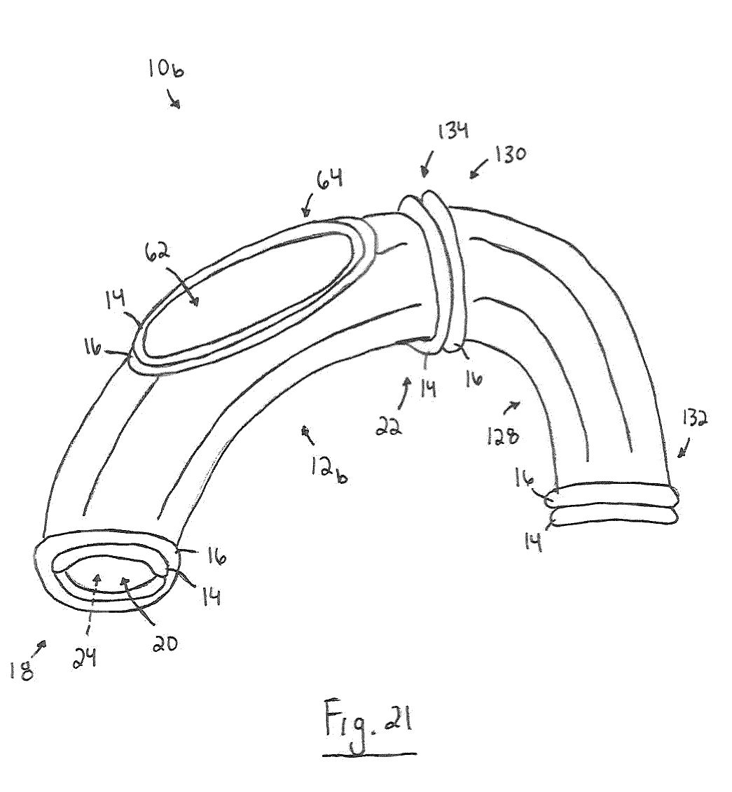

As shown in FIG. 20, the portions of the ascending and descending aorta 142 and 138 can be anastomosed with the first and second sewing rings 14 and 16 at the first distal end portion 18 and the second end portion 132, respectively, in a manner similar or identical as shown in FIGS. 13A-B (described above). Anastomosis of the portions of the ascending and descending aorta 142 and 138 with the first and second sewing rings 14 and 16 at the first distal end portion 18 and the second end portion 132, respectively, provides a double hemostatic effect.

The first, second and third branch members 116, 118, and 120 of the multi-lumen branch graft 80b can then be respectively anastomosed with the brachiocephalic trunk artery 122, the left common carotid artery 124, and the left subclavian artery 126 in a manner similar or identical as shown in FIGS. 13A-B (described above). Although not shown in FIG. 20, the second attachment ring 134 of the vascular graft 10b can then be secured to the aortic arch 136. It will be appreciated that the second attachment ring 134 can also be secured to the ascending aorta (not shown in detail), the aortic root (not shown), or used to secure a mechanical or biological bioprosthetic valve (not shown) in a native cardiac valve (not shown). After the vascular graft 10b is secured in place of the aortic arch aneurysm, the incision in the aortic arch 136 can be closed and the vessels surrounding the vascular graft unclamped so that blood can flow normally through the vascular graft.

From the above description of the invention, those skilled in the art will perceive improvements, changes and modifications. For example, the first and second base portions 34 and 36 can be integrally connected so that the first and second sewing rings 14 and 16 form a single sewing ring (not shown) having a Y-shaped configuration. Such improvements, changes, and modifications are within the skill of the art and are intended to be covered by the appended claims.

Having described the invention, I claim:

1. A vascular graft comprising:
an elongated main body portion having a first distal end portion defining a first opening and a second proximal end portion defining a second opening;
a first sewing ring having a first base portion securely attached to said first distal end portion of said elongated main body portion; and
a second sewing ring having a second base portion securely attached to said first distal end portion at a location proximal to said first sewing ring;
wherein said first and second base portions form a channel for receiving an end portion of a blood vessel.

2. The vascular graft of claim 1, wherein said first and second base portions are spaced apart from one another.

3. The vascular graft of claim 1, wherein said first and second base portions are in contact with one another.

4. The vascular graft of claim 1, wherein said elongated main body portion further comprises a lumen extending between said first distal end portion and said second proximal end portion and being defined by an outer surface and an inner surface.

5. The vascular graft of claim 1, wherein said first distal end portion of said elongated main body portion further includes a distal tip.

6. The vascular graft of claim 4, wherein said first base portion of said of said first sewing ring is securely attached to said outer surface of said elongated main body portion.

7. The vascular graft of claim 5, wherein said first base portion of said of said first sewing ring is securely attached to said distal tip of said elongated main body portion.

8. The vascular graft of claim 1, wherein said elongated main body portion further includes at least one expandable support member operably secured to said inner surface of said lumen.

9. A method for repairing at least a portion of a blood vessel, said method comprising the steps of:
providing a vascular graft, the vascular graft comprising an elongated main body portion having a first distal end portion defining a first opening and a second proximal end portion defining a second opening, a first sewing ring having a first base portion securely attached to the first distal end portion, and a second sewing ring having a second base portion securely attached to the first distal end portion at a location proximal to the first sewing ring, the first and second base portions forming a channel for receiving an end portion of a blood vessel;
determining a placement position for the vascular graft at the portion of the blood vessel to be repaired;
delivering the vascular graft to the portion of the blood vessel to be repaired;
positioning the end portion of the blood vessel in the channel formed by the first and second sewing rings; and
securing the end portion of the blood vessel between the first and second sewing rings so that the lumen of the blood vessel to be repaired and the lumen of the elongated main body portion are in fluid communication with one another.

10. The method of claim 9, wherein said step of securing the end portion of the blood vessel between the first and second sewing rings further includes the steps of:
contacting a portion of the second sewing ring with a portion of the first sewing ring so that the end portion of the blood vessel is sandwiched between the first and second sewing rings; and
sewing the first and second sewing rings together to secure the end portion of the blood vessel therebetween and thereby provide a double hemostatic effect.
